(19) United States
(12) Patent Application Publication
(10) Pub. No.: US 2009/0047742 A1
(43) Pub. Date: Feb. 19, 2009

(54) Inhibition of mRNA Interferase-Induced Apoptosis in BAK-Deficient and BAK- and Bax-Deficient Mammalian Cells

(75) Inventors: Tsutomu Shimazu, Highland Park, NJ (US); Kurt Degenhardt, Montclair, NJ (US); Eileen White, Princeton, NJ (US); Masayori Inouye, New Brunswick, NJ (US)

Correspondence Address: PATENT DOCKET ADMINISTRATOR;LOWENSTEIN SANDLER P.C., 65 LIVINGSTON AVENUE, ROSELAND, NJ, 07068, US

(73) Assignee: UNIVERSITY OF MEDICINE AND DENTISTRY OF NEW JERSEY, Somerset, NJ (US)

(21) Appl. No.: 12/064,070

(22) PCT Filed: Aug. 22, 2006
(86) PCT No.: PCT/US06/32981

§ 371 Date: Aug. 18, 2008

Related U.S. Application Data
(60) Provisional application No. 60/710,900, filed on Aug. 24, 2005.
(60) Provisional application No. 60/817,273, filed on Jun. 29, 2006.

(51) Int. Cl. C12N 15/87; C12N 5/06

(52) U.S. Cl. 435/455; 435/325

(57) ABSTRACT

Ribonucleases, antibiotics, bacterial toxins and viruses inhibit protein synthesis, which results in apoptosis in mammalian cells. How the BCL-2 family of proteins regulates apoptosis in response to shutoff of protein synthesis is not known. According to the present invention, an Escherichia coli toxin MazF inhibited protein synthesis by cleavage of cellular mRNA, and induced apoptosis in mammalian cells. MazF-induced apoptosis required proapoptotic BAK and its upstream regulator, the proapoptotic BH3-only protein NBK/BIK, but not BIM, PUMA or NOXA. Furthermore, NBK/BIK- or BAK-deficient cells were resistant to cell death induced by pharmacologic inhibition of translation and by virus-mediated shutoff of protein synthesis. Thus, the BH3-only protein NBK/BIK is the apical regulator of a BAK-dependent apoptotic pathway in response to shutoff of protein synthesis. Although NBK/BIK is dispensable for development, it is the BH3-only protein targeted for inactivation by viruses, suggesting that it plays a role in pathogen/toxin response through apoptosis activation.

### CROSS REFERENCE TO RELATED APPLICATIONS

This application claims the benefit of priority to U.S. provisional application No. 60/817,273 (filed Jun. 29, 2006), and entitled “NBK/BIK Regulates BAK-mediated Apoptosis Induced by Inhibition of Protein Synthesis,” and U.S. provisional application No. 60/710,900 (filed Aug. 24, 2005) and entitled “Inhibition of mRNA interferase-induced apoptosis in BAK-deficient and BAK- and BAX-deficient mammalian cells,” the contents of which are incorporated by reference herein.

### FIELD OF THE INVENTION

The present invention relates to regulation of mRNA interferase-induced apoptosis in mammalian cells.

### BACKGROUND OF THE INVENTION

Apoptosis is a genetically coordinated and conserved cell death process in organisms from C. elegans to vertebrates (Adams, J. M., Genes Dev. 17: 2481-2495 (2003); Danial, N. N., and Korsmeyer, S. J. Cell 116: 205-219. (2004)). It not only is essential for successful crafting of complex multicellular tissues during embryonic development and for maintenance of normal cellular homeostasis in adult organisms, but also is needed for elimination of cells damaged by stress or pathogen infection (White, E. Cell Death Differ. 13: 1-7 (2006)). A critical point of apoptosis regulation is controlled by members of the Bcl-2 family. The Bcl-2 family of proteins can be divided into three different subclasses based on conservation of Bcl-2 homology (BH1-4) domains: multidomain anti-apoptotic proteins (Bcl-2, Bcl-X_{L}, Mcl-1, Bcl-W and Bfl-1/A1), multidomain proapoptotic proteins (BAX and BAK), and BH3-only proapoptotic proteins (BID, BAD, BIM, PUMA, NOXA and NBK/BIK) ((Adams, J. M., Genes Dev. 17: 2481-2495 (2003); Danial, N. N., and Korsmeyer, S. J. Cell 116: 205-219. (2004); Gelinas, C., and White, E. Genes Dev. 19: 1263-1268 (2005); Willis, S. N., and Adams, J. M. Curr. Opin. in Cell Biol. 17: 1-9 (2005)). Notably, BH3-only proteins are not able to kill cells that lack BAX and BAK, indicating that BH3-only proteins are upstream of, and are dependent upon, BAX and BAK (Zong, W. Y, et al., Genes Dev. 15: 1481-1486 (2001)).

The proapoptotic BH3-only proteins are the most apical mediators of death induced by cytokine deprivation, activated oncogenes, DNA damage, chemotherapy and γ-irradiation. For example, BID is a critical mediator of apoptosis mediated by death receptor signaling (Li, H., et al., Cell 94: 491-501 (1998); Luo, X., et al., Cell 94: 481-490. (1998)). BIM is the determinant of taxane responsiveness (Bouillet, P., et al., Science 286: 1735-1738 (1999); . Tan, T. T., et al., Cancer Cell 7: 227-238 (2005)), PUMA and NOXA are central mediators of p53-induced apoptosis (Jefferes, J. R., et al., Cancer Cell 4: 321-328 (2003); Shibue, T., et al Genes Dev. 17: 2233-2238. (2003); Villunger, et al., Science 302: 1036-1038 (2003)), and BAD regulates apoptosis mediated by growth factors/cytokines signaling (Datta, S. R., et al., Mol. Cell 6: 41-51 (2000); Datta, S. R. et al. Dev. Cell 3: 631-643. (2002)). In contrast, the cellular responses to trigger specifically the NBK/BIK-mediated apoptotic pathway are poorly characterized.

As in mammalian cells, bacterial cells also regulate cell death. In E. coli cells, growth inhibition and subsequent cell death are mediated through a unique genetic system called “addiction modules” or “toxin-antitoxin modules”, which consist of a pair of genes encoding two components, one for a stable toxin and the other for an unstable antitoxin (Engelberg-Kulka et al., Trends Microbiol. 12: 66-71 (2004); Gerdes K. et al. Nat. Rev. Microbiol. 3: 371-382 (2005)). The antitoxin and toxin usually are co-expressed in the same operon (referred to as an “addiction module” or “antitoxin-toxin system”), and their expression and function are negatively autoregulated either by the complex of antitoxin and toxin or by antitoxin alone. When the co-expression of antitoxin and toxin is inhibited, the antitoxin is rapidly degraded by a specific protease, enabling the toxin to act on its target. Such a genetic system for bacterial cell growth inhibition has been reported in a number of E. coli extrachromosomal elements (Gerdes, K. et al. Nat. Rev. Microbiol. 3: 371-382 (2005)).

One of the addiction modules on the E. coli chromosome, the mazEF system, consists of two adjacent genes, mazE and mazF, located downstream from the relA gene (Aizenman, E., et al., Proc. Natl. Acad. Sci. USA 93: 6059-6063 (1996)). MazF is a sequence specific endoribonuclease that specifically cleaves single-stranded RNAs (ssRNAs) at ACA sequences. An “endonuclease” is one of a large group of enzymes that specifically cleaves nucleic acids at positions within a nucleic acid chain. Endoribonucleases or ribonucleases are specific for RNA. MazF is referred to as an mRNA interferase since its primary target is messenger RNA (mRNA) in vivo. MazF is a stable toxin whereas MazE is a labile antitoxin that is quickly degraded by ChpPA, an ATP-dependent serine protease (Aizenman, E., et al., Proc. Natl. Acad. Sci. USA 93: 6059-6063 (1996)). It recently has been demonstrated that MazF is a sequence-specific endoribonuclease which specifically cleaves E. coli mRNA at the ACA triplet sequence to block de novo protein synthesis, resulting in cell growth arrest and subsequent bacterial cell death (Zhang, Y., et al., Mol. Cell 12: 913-923 (2003)). Furthermore, it has been shown that MazE is responsible for antagonizing the endoribonuclease activity of MazF (Zhang, Y, et al., Mol. Cell 12: 913-923 (2003). The purpose of this addiction module is to provide a competitive growth advantage to the bacteria that encode it.

As in bacteria, inhibition of protein synthesis in mammalian cells induced by ribonuclease-mediated RNA cleavage, translation silencing with antibiotics, or pathogen infection leads to programmed cell death. In response to viral infection, interferons activate RNase L that cleaves 18S and 28S ribosomal RNA, which inhibits protein synthesis, eventually inducing apoptosis mediated by cytochrome c release and caspase-3 activation to eliminate virus-infected cells (Silverman, R. H., Biochemistry 42: 1805-1812 (2003); Xiang, Y., et al., Cancer Res. 63: 6795-6801 (2003). Virus-produced double-strand RNA (dsRNA) activates RNA-activated protein kinase (PKR) which phosphorylates eukaryotic initiation factor 2 (eIF-2) thereby inhibiting mRNA translation, leading to apoptosis (Gil, J., and Esteban, M. Apoptosis 5: 107-114 (2000)). In turn, viruses have evolved mechanisms to evade these and other host defenses by enabling viral but not host protein synthesis (Barzilai, A., et al., J. Virol. 80: 505-513 (2005)) and through inhibition of apoptosis (White, E., Cell Death Differ. 13: 1-7 (2006); Roulston, A., et al., Annu. Rev. Microbiol. 53: 577-628. (1999)). Adenovirus, for example, encodes factors that block interferon-mediated gene expression, inhibit PKR activation, and prevent apoptosis (Roulston, A., et al., Annu. Rev. Microbiol. 53: 577-628. (1999); Cuconati, A., and White, E. Genes Dev. 16: 2465-2478. (2002)). This allows viral but not cellular protein synthesis without cell death. Finally, antibiotics, such as cycloheximide (CHX), puromycin and emetin, are part of the anti-bacterial arsenal used to inhibit and kill pathogens by targeting protein synthesis by various mechanisms (Meijerman, I., et al., Toxicol. Appl. Pharmacol. 156: 46-55. (1999)). Although inhibition of protein synthesis by various means is a common weapon to gain a selective advantage, and is known to activate the apoptotic response in mammalian cells, the pathway utilized to activate apoptosis is not known.

It now has been demonstrated that the bacterial toxin, MazF, induces striking degradation of cellular mRNA and inhibition of protein synthesis in mammalian cells just as in bacteria. MazF expression in mammalian cells causes caspase-3 activation and poly (ADP-ribose) polymerase (“PARP”) cleavage, which are hallmarks of apoptotic cell death, all of which were blocked by the antitoxin MazE. Interestingly, expression of MazF in immortalized baby mouse kidney (“iBMK”) cells deficient for bax and/or bak, or BH3-only proapoptotic genes (puma, bim, noxa and nbk/bik) revealed that NBK/BIK and BAK were required for apoptosis induced by MazF. Moreover, BAX and BAK, BAK or NBK/BIK-deficiency conferred resistance to cell death induced by protein synthesis inhibition by cycloheximide and shutoff of protein synthesis induced by viral infection. As shutoff of protein synthesis is often a cellular response to pathogens, this signifies that an NBK/BIK and BAK-specific apoptotic pathway may control this process.

### BRIEF DESCRIPTION OF THE FIGURES

FIG. 1 shows that MazF expression causes degradation of cellular mRNAs in mammalian cells. (A) Northern blot analysis of human GAPDH and β-actin. Total RNA from Tet-treated or -untreated T-Rex-293 cells at the indicated time points was probed with ³²P-labeled human GAPDH and β-actin cDNA. 28S and 18S ribosomal RNAs were visualized by agarose-formaldehyde gel electrophoresis followed by ethidium bromide staining. (B) Quantification of mRNA levels. Human GAPDH and β-actin mRNA levels were quantified by real-time RT-PCR. Relative amounts of mRNA were calculated from the fluorescence signal in the 24-, 48- and 72-hours samples as compared with the corresponding 0-hour sample.

FIG. 2 shows that MazF inhibits protein synthesis in mammalian cells. (A) ³⁵S-methionine incorporation in T-Rex-293 cells. ³⁵S-methionine-labeled total protein from Tet-treated T-Rex-293 cells at the indicated time points was subjected to SDS-PAGE and autoradiography (left) and stained with Coomassie blue (right). (B) Quantification of ³⁵S-methionine-labeled proteins. Protein bands on the gel in (A) were scanned by Phosphoimager STORM 860 (Molecular dynamics) and signal intensity was calculated.

FIG. 3 shows that MazF induces apoptotic cell death in mammalian cells. (A) Phase contrast photographs of Tet-treated or -untreated T-Rex-293 cells (magnification 100×). (B) Viability analysis of T-Rex-293 cells in (A). Tet-treated or -untreated T-Rex-293 cells at the indicated time points were subjected to trypan blue exclusion. Viability was represented as a percent of total cells at time 0. (C) Representative illustration of propidium iodide labeling measured by FACS in Tet-treated T-Rex-293 cells. (D) Western blot analysis with lysates from T-Rex-293 cells. Whole cell lysates from Tet-treated or staurosporine-treated T-Rex-293 cells at the indicated time points was immunoblotted with an anti-active caspase-3 antibody (top), anti-PARP antibody (middle) and anti-actin antibody (bottom).

FIG. 4 shows that levels of BCL-2 family proteins remain constant during MazF induction. Whole cell lysates from Tet-treated T-Rex-293 cells was immunoblotted with antibodies that specifically recognize anti-apoptotic and proapoptotic proteins indicated in the figure.

FIG. 5 shows that BAK function is required for MazF-induced apoptosis. (A) Viability of iBMK cells transiently expressing MazF. W2, D3, X2 and K1 cells transiently co-expressing LacZ and MazF were subjected to a β-galactosidase assay at 48 hours post-transfection. β-Galactosidase positive blue cells were calculated as its percentage of total cells. (B) Immunofluorescence of activated caspase-3 in iBMK cells. W2, D3, X2 or K1 cells transiently co-expressing LacZ and MazF were co-stained with anti-Xpress and anti-active caspase-3 antibody. FITC (green) and rhodamine (red) stain represent cells expressing LacZ and activated caspase-3, respectively. Numbers represents the percentage of activated-caspase-3 positive cells. White arrows indicate the corresponding activated-caspase-3 positive cells from the matching FITC-stained cells. (C) Viability of iBMK cells transiently co-expressing MazF and MazE. W2, D3, X2 and K1 cells transiently co-expressing LacZ and MazF and/or MazE were subjected to a β-galactosidase assay. β-Galactosidase positive blue cells were calculated as described above.

FIG. 6 shows that NBK/BIK mediates MazF or CHX-induced cell death. (A) Viability of iBMK cells transiently expressing MazF. nbk/bik^{-/-}, bim^{-/-}, noxa^{-/-} or puma^{-/-} iBMK cells co-expressing LacZ and MazF were subjected to a β-galactosidase assay. β-Galactosidase positive blue cells were calculated as its percentage of total cells. (B) and (C), Viability of CHX-treated iBMK cells. W2, D3, X2 and K1 cells (B), and nbk/bik^{-/-}B, bim^{-/-}, noxa^{-/-} or puma^{-/-} cells (C) treated with CHX were subjected to an MTT assay. (D) and (E), Viability of TNF-□/CHX- and paclitaxel-treated iBMK cells. W2, D3 and three independent nbk/bik^{-/-} cell lines (A, B and C) treated with TNF-α/CHX (0.05 μg/ml) (D) and paclitaxel (E) were subjected to an MTT assay.

FIG. 7 shows that NBK/BIK mediates adenovirus-induced apoptosis. (A) Phase contrast photographs of adenovirus-infected iBMK cells (magnification 100×). (B) Western blot analysis with lysates from viral infected iBMK cells. Whole cell lysates from mock-, Ad5d/309- or Ad5d/337-infected W2, D3 and nbk/bik^{-/-} B cells were immunoblotted with an anti-active caspase-3 antibody (top), anti-E1A antibody (middle) and anti-actin antibody (bottom). (C) Apoptosis pathway induced by shutoff of protein synthesis.

### SUMMARY OF THE INVENTION

The present invention provides a method of selectively regulating apoptosis in a mammalian cell, the method comprising the steps: (a) preparing a first expression vector comprising an isolated nucleic acid sequence encoding an mRNA interferase polypeptide, a derivative of the mRNA interferase polypeptide, or a fragment of the mRNA interferase polypeptide; (b) preparing a second expression vector comprising an isolated nucleic acid sequence encoding an mRNA interferase antagonist polypeptide, a derivative of the mRNA interferase antagonist polypeptide, or a fragment of the mRNA interferase antagonist polypeptide; (c) introducing the first expression vector into the mammalian cell, (d) optionally introducing the second expression vector into the mammalian cell; (e) selectively inducing expression of the first expression vector encoding the mRNA interferase polypeptide, the derivative of the mRNA interferase polypeptide, or the fragment of the mRNA interferase polypeptide, thereby inducing apoptosis in the cell, and (f) optionally selectively inducing expression of the second expression vector encoding the mRNA interferase antagonist polypeptide, the derivative of the mRNA interferase antagonist polypeptide, or the fragment of the mRNA interferase antagonist polypeptide, thereby inhibiting apoptosis in the cell. In one embodiment, the first expression vector and the second expression vector each further comprise at least one regulatory sequence. In another embodiment, the at least one regulatory sequence is at least one inducible promoter. In another embodiment, the at least one inducible promoter in the first expression vector is operably linked to the nucleic acid sequence encoding the mRNA interferase polypeptide, the derivative of the mRNA interferase polypeptide, or the fragment of the mRNA interferase polypeptide. In another embodiment, the at least one inducible promoter in the second expression vector is operably linked to the nucleic acid sequence encoding the mRNA interferase antagonist polypeptide, the derivative of the mRNA interferase antagonist polypeptide, or the fragment of the mRNA interferase antagonist polypeptide. In another embodiment, the mRNA interferase polypeptide, derivative of the mRNA interferase polypeptide, or fragment of the mRNA interferase polypeptide when expressed in the cell recognizes an at least one first mRNA interferase recognition sequence in cellular messenger RNA. In another embodiment, the at least one first mRNA interferase recognition sequence is adenine-cytosine-adenine. In another embodiment, the mRNA interferase polypeptide is a prokaryotic polypeptide. In another embodiment, the mRNA interferase polypeptide is MazF. In another embodiment, the mRNA interferase antagonist polypeptide is a prokaryotic polypeptide. In another embodiment, the mRNA interferase antagonist polypeptide is MazE. In another embodiment, the target mammalian cell is Bak-deficient. In another embodiment, the target mammalian cell is Bak- and Bax-deficient. In another embodiment, the target mammalian cell is a tumor cell. In another embodiment, the target mammalian cell is infected by a pathogen. In another embodiment, the pathogen is a bacterium, a virus, a fungus, a parasite or a prion. In another embodiment, the target mammalian cell is a stem cell. In another embodiment, the target mammalian cell is a differentiated cell. In another embodiment, the differentiated cell is a muscle cell, a kidney cell, a lung cell, a thyroid cell, a pancreatic cell, a blood cell, a nerve cell, a glial cell, or a sensory cell. In another embodiment, the target mammalian cell is an immune cell. In another embodiment, the target mammalian cell is a genetically damaged cell. In another embodiment, the target mammalian cell is a toxin-damaged cell.

In another aspect, the present invention provides a method of maintaining an isolated nucleic acid sequence encoding an mRNA interferase polypeptide, a derivative of the mRNA interferase polypeptide, or a fragment of the mRNA interferase polypeptide in a mammalian cell, the method comprising the steps: (a) preparing an expression vector comprising an isolated nucleic acid sequence encoding an mRNA interferase polypeptide, a derivative of the mRNA interferase polypeptide, or a fragment of the mRNA interferase polypeptide; and (b) introducing the expression vector into the mammalian cell, wherein at least one apoptotic pathway of the mammalian cell is blocked. In one embodiment, the method further comprising the step of (c) inducing the expression of the mRNA interferase polypeptide, the derivative of the mRNA interferase polypeptide, or a fragment of the mRNA interferase polypeptide after step (b). In another embodiment, the expression vector further comprises at least one regulatory sequence. In another embodiment, the at least one regulatory sequence is at least one inducible promoter which is operably linked to the nucleic acid sequence encoding the mRNA interferase polypeptide, the derivative of the mRNA interferase polypeptide, or the fragment of the mRNA interferase polypeptide. In another embodiment, the mRNA interferase polypeptide, derivative of the mRNA interferase polypeptide, or fragment of the mRNA interferase polypeptide, when expressed in the cell, recognizes an at least one mRNA interferase recognition sequence in cellular messenger RNA. In another embodiment, the at least one mRNA interferase recognition sequence is adenine-cytosine-adenine. In another embodiment, the mRNA interferase polypeptide is a prokaryotic polypeptide. In another embodiment, the mRNA interferase polypeptide is MazF. In another embodiment, the mammalian cell is BAK deficient, NBK/BIK deficient, or BAK deficient and NBK/BIK deficient.

In another aspect, the present invention provides a mammalian cell harboring an isolated nucleic acid sequence encoding an mRNA interferase polypeptide, a derivative of the mRNA interferase polypeptide, or a fragment of the mRNA interferase polypeptide, wherein at least one apoptotic pathway of said mammalian cell is blocked. In one embodiment, the mammalian cell is transduced by an expression vector comprising an isolated nucleic acid sequence encoding an mRNA interferase polypeptide, a derivative of the mRNA interferase polypeptide, or a fragment of the mRNA interferase polypeptide. In another embodiment, the expression vector further comprises at least one regulatory sequence. In another embodiment, the at least one regulatory sequence is at least one inducible promoter which is operably linked to the nucleic acid sequence encoding the mRNA interferase polypeptide, the derivative of the mRNA interferase polypeptide, or the fragment of the mRNA interferase polypeptide. In another embodiment, the mRNA interferase polypeptide, derivative of the mRNA interferase polypeptide, or fragment of the mRNA interferase polypeptide, when expressed in the cell, recognizes an at least one mRNA interferase recognition sequence in cellular messenger RNA. In another embodiment, the at least one mRNA interferase recognition sequence is adenine-cytosine-adenine. In another embodiment, the mRNA interferase polypeptide is a prokaryotic polypeptide. In another embodiment, the mRNA interferase polypeptide is MazF. In another embodiment, the mammalian cell is BAK deficient, NBK/BIK deficient, or BAK deficient and NBK/BIK deficient.

### DETAILED DESCRIPTION OF THE INVENTION

The following definitions set forth the parameters of the present invention.

The abbreviation “ACA” refers to the sequence Adenine-Cytosine-Adenine.

The term “apoptosis” as used herein refers to a normal cellular process involving a programmed series of events by which individual cells perish in an orderly, highly controlled manner without releasing harmful substances into the surrounding area. It is distinguished from necrosis, the other form of cell death, which is a degenerative phenomenon that follows irreversible injury. Apoptotic cells may be characterized by specific morphologic and biochemical changes orchestrated by a family of cysteine proteases known as caspases. Morphologically, apoptosis involves condensation of the nuclear chromatin and cytoplasm, fragmentation of the nucleus, and budding of the whole cell to produce membrane-bounded bodies in which organelles are initially intact. These bodies are disposed of by adjacent cells without inflammation. Biochemically, in apoptosis, there is distinctive internucleosome cleavage of DNA, unlike the random DNA degradation observed in necrosis.

At the molecular level, apoptosis is tightly regulated. There are two main pathways leading to apoptotic cell death, namely the death receptor pathway (also called the extrinsic pathway) and the mitochondrial (intrinsic) pathway. The death receptor pathway is believed to involve the interaction of a death receptor, i.e., one of at least five transmembrane receptors belonging to the TNF (tumor necrosis factor)/NGF (nerve growth factor)-receptor superfamily (reviewed by Timmer et al., J. Pathol. 196(2): 125-34 (2002)) such as the tumor necrosis factor (TNF) receptor-1 or the membrane-bound cell-surface Fas receptor, with its ligand.

Proapoptotic and antiapoptotic members of the Bcl-2 family are thought to regulate the second or mitochondrial pathway, which depends on the participation of mitochondria. The mitochondrial pathway is mediated by mitochondrial membrane permeabilization and the release of cytochrome c. Cellular stress induces pro-apoptotic Bcl-2 family members to translocate from the cytosol to the mitochondria, where they induce the release of cytochrome c, while the anti-apoptotic Bcl-2 proteins work to prevent cytochrome c release from mitochondria, and thereby preserve cell survival. Once in the cytoplasm, cytochrome c catalyzes the oligomerization of apoptotic protease activating factor-1, thereby promoting the activation of procaspase-9, which then activates procaspase-3. BAX and/or BAK are required for mitochondrial membrane permeability and function of the intrinsic apoptotic pathway. In addition to their mitochondrial related functions, BAX and BAK also localize to the endoplasmic reticulum (ER) and initiate a parallel pathway of caspase (caspase 12) activation and apoptosis. (Zong et al., J. Cell Biol. 162(1):59-69 (2003)).

Alternatively, ligation of death receptors, like tumor necrosis factor receptor-1 and the Fas receptor, causes the activation of procaspase-8. The mature caspase may either directly activate procaspase-3 or cleave the pro-apoptotic BH3-only protein BID, which subsequently induces cytochrome c release. Most cells use BID-mediated BAX and BAK activation to amplify the extrinsic pathway.

The end result of either pathway is caspase activation and the cleavage of specific cellular substrates, resulting in the morphologic and biochemical changes associated with the apoptotic phenotype.

The term “abnormal apoptosis” as used herein refers to excessive apoptosis or to a failure of apoptosis. Abnormal apoptosis may be deleterious and can cause or contribute to various diseases, disorders, syndromes, conditions or injuries. For example, without limitation, abnormal apoptosis has been implicated in cancer, autoimmune disorders, neurodegenerative disorders, including Huntington's disease, Alzheimer's disease, and stroke.

The term “disease” or “disorder” as used herein refers to an impairment of health or a condition of abnormal functioning. The term “syndrome,” as used herein, refers to a pattern of symptoms indicative of some disease or condition. The term “injury,” as used herein, refers to damage or harm to a structure or function of the body caused by an outside agent or force, which may be physical or chemical. The term “condition”, as used herein, refers to a variety of health states and is meant to include disorders or diseases caused by any underlying mechanism, disorder or injury.

The present invention provides methods for selective (meaning to choose in preference to another or others, pick out) regulation of apoptosis in a target mammalian cell. In some embodiments the methods described herein are used to induce apoptosis in a target mammalian cell. In some embodiments, the methods described herein are used to inhibit apoptosis in a target mammalian cell. In some embodiments, the target mammalian cell can be infected by a pathogen (meaning an agent that disrupts the normal physiology of the cell and causes symptoms of disease), such as a virus, a bacterium, a fungus, a parasite, or a prion. In some embodiments, the target mammalian cell is a stem cell. In some embodiments, the target mammalian cell is a differentiated cell. In some embodiments, the target mammalian cell is a tumor cell, including, but not limited to, a metastatic tumor cell. In some embodiments, the target mammalian cell is a genetically damaged cell, i.e., one whose type of cellular damage results in changes that are passed along to its offspring. In some embodiments, the target cell is a toxin-damaged cell, meaning a cell exposed to and damaged by a naturally occurring or synthetic substance that is toxic [meaning poisonous, carcinogenic, or otherwise directly harmful to life] when introduced into living tissue. In some embodiments, the target cell is an immune cell having abnormal apoptosis and the methods of the present invention are used to restore normal immune system function.

The terms “antagonist” and “inhibitor” are used interchangeably herein to refer to an agent that prevents, reduces, blocks, neutralizes or counteracts the effects of another agent.

The term “Bcl-2 protein” (“B-cell lymphoma 2 protein”) refers to a family of transmembrane proteins that regulate the activity of one or more components of the apoptotic pathway. Without being limited by theory, their main mechanism of action is thought to be the regulation of mitochondrial membrane permeability. Some members of the family are pro-apoptotic, while others are anti-apoptotic. The term “proapoptotic” as used herein refers to activities, components, or effects that promote cell death. Pro-apoptotic members of the Bcl-2 superfamily are believed to increase mitochondrial membrane permeability. The term “anti-apoptotic” as used herein refers to activities, components and effects that inhibit apoptosis at least in part by opposing this increase in mitochondrial membrane permeability. The BCL-2 family is classed into three subfamilies, which share some regions of homology known as BCL-2 Homology (or BH) regions. Starting from the amino terminal end (N), and moving from the left to the right towards the C-terminal end (“C), the BH regions are arranged as follows:

N-BH4-BH3-BH1-BH2-TM-C,

where TM refers to the transmembrane spanning region. The Bcl-2 subfamily includes, without limitation, Bcl-2, Bcl-xL, Bcl-w and MCL-1. They are anti-apoptotic, and promote cell survival.

The term “Mcl-1” (myeloid cell leukemia sequence 1) as used herein refers to a Bcl-2-related antiapoptotic protein originally isolated from human myeloid leukemia cells.

BH3-only proteins (1) activate BAX and BAK, and (2) antagonize anti-apoptotic proteins like BCL-2 to induce apoptosis. The BAX subfamily of Bcl-2 proteins includes BAX and BAK. These are pro-apoptotic and promote cell death. They show sequence homology with the Bcl-2 subfamily in the BH1, BH2 and BH3 regions, but not the BH4 region. Due to its extensive sequence homology with Bcl-2, BAX can form heterodimers with Bcl-2. Homo- or heterodimers of BAX repress the antiapoptotic activity of Bcl-2.

The BH3-only subfamily includes BAD and BID proteins, which are pro-apoptotic proteins that promote cell death. They only share sequence homology with the Bcl-2 subfamily in the BH3 region. BID also lacks the transmembrane-spanning region.

The term “Bim” refers to a proapoptotic member of the Bcl-2 family of proteins that plays an essential role in the mitochondrial pathway of apoptosis through activation of the BH1-3 multidomain protein BAX or BAK.

“NBK/Bik” signifies “natural born killer/Bcl-2 interacting killer” and refers to a proapoptotic Bcl-2-related protein.

The term “PUMA” (“p53-Upregulated Modulator of Apoptosis”) refers to a proapoptotic BH3-only transcriptional target of p53 that functions downstream of p53 and in p53-deficient cells. p53 is a cell cycle related transcription factor and tumor suppressor that promotes transcription of genes that induce cell cycle arrest or apoptosis in response to DNA damage or other cell stresses. Most evidence does not support PUMA binding to p53 as a mechanism of apoptosis induction.

The following table (modified from Antonsson, Cell Tissue Res., 306(3): 347-61 (2001) and Zhang et al. Hu Molec. Genet. 10(21): 2329-39 (2001)) is a summary of the known bcl-2 protein family:

The Bcl-2 Family of Proteins

| Anti-apoptotic | Pro-apoptotic |
|---|---|
| Bcl-2 | BAX |
| Bcl-XL | BAK |
| Bcl-w | Bok |
| Mcl-1 | Bcl-XS |
| BOO/DIVA | BID |
| A1/Bfl-1 | Bad |
| NR-13 | Bik/Nbk |
| Bcl2-L-10 | Bim/Bod |
| | Blk |
| | Hrk |
| | Nix |
| | BNip3 |
| | Noxa |
| | PUMA |
| | Bcl-rambo |

The term “caspases” as used herein refers to a family of cysteine proteases that selectively cleave proteins at sites just C-terminal to aspartate residues and are responsible for the breakdown of the cell during apoptosis by cleaving numerous cellular proteins. Caspases are synthesized as inactive procaspases that are later activated by proteolytic cleavage into active caspases. Pro-apoptotic regulators (e.g., BAX) promote caspase activation.

The term “cDNA” refers to a single stranded complementary or copy DNA synthesized from an mRNA template using the enzyme reverse transcriptase. The single-stranded cDNA often is used as a probe to identify complementary sequences in DNA fragments or genes of interest.

As used herein the terms “differentiated cell” or “differentiated cells” refer to cells that are specialized for a particular function and do not maintain the ability to generate other kinds of cells, or revert back to a less specialized cell. They include, without limitation, muscle cells (i.e., cells that are specialized to produce mechanical force), tubule cells of the kidney, lung (alveolar) cells, thyroid cells, pancreatic cells, blood cells (including, but not limited to, erythrocytes, leukocytes (including lymphocytes, macrophages and neutrophils) and platelets), glial cells, nerve cells or neurons (i.e., cells that are specialized for communication), and sensory cells (meaning cells that detect external stimuli, e.g., hair cells of the inner ear, rod cells in the retina of the eye).

As used herein, the terms “encode”, “encoding” or “encoded”, with respect to a specified nucleic acid, refers to information stored in a nucleic acid for translation into a specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons UUA, UUG, CUU, CUC, CUA, and CUG all encode the amino acid leucine. Thus, at every position where a leucine is specified by a codon, the codon can be altered to any of the corresponding codons described without altering the encoded polypeptide. Such nucleic acid variations are “silent variations” and represent one species of conservatively modified variation. Every nucleic acid sequence herein which encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is within the scope of the present invention.

The present invention includes active portions, fragments, derivatives, mutants, and functional variants of mRNA interferase polypeptides to the extent such active portions, fragments, derivatives, and functional variants retain any of the biological properties of the mRNA interferase. An “active portion” of an mRNA interferase polypeptide means a peptide that is shorter than the full length polypeptide, but which retains measurable biological activity. A “fragment” of an mRNA interferase means a stretch of amino acid residues of at least five to seven contiguous amino acids, often at least about seven to nine contiguous amino acids, typically at least about nine to thirteen contiguous amino acids and, most preferably, at least about twenty to thirty or more contiguous amino acids. A “derivative” of an mRNA interferase or a fragment thereof means a polypeptide modified by varying the amino acid sequence of the protein, e.g., by manipulating the nucleic acid encoding the protein or by altering the protein itself. Such derivatives of the natural amino acid sequence may involve insertion, addition, deletion, or substitution of one or more amino acids, and may or may not alter the essential activity of the original mRNA interferase.

Glyceraldehyde 3-phosphate dehydrogenase is abbreviated herein as GAPDH.

The term “gene” refers to an ordered sequence of nucleotides located in a particular position on a segment of DNA that encodes a specific functional product (i.e, a protein or RNA molecule). It can include regions preceding and following the coding DNA as well as introns between the exons.

The term “immune cell” as used herein refers to cells of the immune system that prompt, alert, facilitate, activate, surround, kill, clean up, or synthesize and secrete messengers, regulators or helpers in the process of defending a subject against invaders, including, but not limited to, scavenger cells (e.g., monocytes/macrophages), natural killer (NK) cells, and lymphocytes (including, but not limited to, B cells and T cells).

The term “induce” or “inducible” refers to a gene or gene product whose transcription or synthesis is increased by exposure of the cells to an inducer or to a condition.

The terms “inducer” or “inducing agent” refer to a low molecular weight compound or a physical agent that associates with a repressor protein to produce a complex that no longer can bind to the operator.

The terms “introduced”, “transfection”, “transformation”, “transduction” in the context of inserting a nucleic acid into a cell, include reference to the incorporation of a nucleic acid into a prokaryotic cell or eukaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is substantially free from components that normally accompany or interact with it as found in its naturally occurring environment. The isolated material optionally comprises material not found with the material in its natural environment; or, if the material is in its natural environment, the material has been synthetically (non-naturally) altered by deliberate human intervention. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

The term “MazE” as used herein refers to the general class of antitoxins that antagonize the endoribonuclease activity of MazF and active fragments and derivatives thereof having structural and sequence homology thereto consistent with the role of MazF polypeptides in the present invention.

The term “MazF” as used herein refers to the general class of endoribonucleases, to the particular enzyme bearing the particular name and active fragments and derivatives thereof having structural and sequence homology thereto consistent with the role of MazF polypeptides in the present invention.

The family of enzymes encompassed by the present invention is referred to as “mRNA interferases”. It is intended that the invention extend to molecules having structural and functional similarity consistent with the role of this family of enzymes in the present invention.

As used herein, the term “nucleic acid” or “nucleic acid molecule” includes any DNA or RNA molecule, either single or double stranded, and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. Unless otherwise limited, the term encompasses known analogues.

The term “operator” refers to the region of DNA that is upstream (5′) from a gene(s) and to which one or more regulatory proteins (repressor or activator) bind to control the expression of the gene(s).

As used herein, the term “operon” refers to a functionally integrated genetic unit for the control of gene expression. It consists of one or more genes that encode one or more polypeptide(s) and the adjacent site (promoter and operator) that controls their expression by regulating the transcription of the structural genes. The term “expression operon” refers to a nucleic acid segment that may possess transcriptional and translational control sequences, such as promoters, enhancers, translational start signals, polyadenylation signals, terminators, and the like, and which facilitate the expression of a polypeptide coding sequence in a host cell or organism.

The phrase “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The abbreviation “PCR” refers to polymerase chain reaction, which is a technique for amplifying the quantity of DNA, thus making the DNA easier to isolate, clone and sequence. See, e.g., U.S. Pat. Nos. 5,656,493, 5,33,675, 5,234,824, and 5,187,083, each of which is incorporated herein by reference.

As used herein the term “promoter” includes reference to a region of DNA upstream (5′) from the start of transcription and involved in recognition and binding of RNA polymerase and other proteins to initiate transcription. The term “inducible promoter” refers to the activation of a promoter in response to either the presence of a particular compound, i.e., the inducer or inducing agent, or to a defined external condition, e.g., elevated temperature.

The term “regulate” as used herein refers to the act of inhibiting, promoting, controlling, managing, directing, or adjusting by some standard or principle or the state of being inhibited, promoted, controlled, managed, directed, or adjusted.

The term “repressor” includes a protein or agent that binds to a specific DNA sequence (the operator) upstream from the transcription initiation site of a gene or operon that can regulate a gene by turning it on and off.

The term “ribosomal RNA” (rRNA) refers to the central component of the ribosome, the protein manufacturing machinery of all living cells. These machines self-assemble into two complex folded structures (the large and the small subunits) in the presence of a plurality of ribosomal proteins. In bacteria, Archaea, mitochondria, and chloroplasts, a small ribosomal subunit contains the 16S rRNA, where the S in 16S represents Svedberg units; the large ribosomal subunit contains two rRNA species (the 5S and 23S rRNAs). Bacterial 16S, 23S, and 5S rRNA genes are typically organized as a co-transcribed operon. There may be one or more copies of the operon dispersed in the genome. Eucaryotic cells generally have many copies of the rRNA genes organized in tandem repeats. The 18S rRNA in most eukaryotes is in the small ribosomal subunit, and the large subunit contains three rRNA species (the 5S, 5.8S and 25S/28S rRNAs).

The term “total RNA” includes messenger RNA (“mRNA”, the RNA that carries information from DNA to the ribosome sites of protein synthesis in the cell where it is translated into protein), transfer RNA (“tRNA”, a small RNA chain that transfer a specific amino acid to a growing polypeptide chain during protein translation; ribosomal RNA (“rRNA”), and noncoding RNA (also known as RNA genes or small RNA, meaning genes that encode RNA that is not translated into protein).

The term “sodium dodecyl sulfate-polyacrylamide gel electrophoresis” is abbreviated SDS-PAGE.

As used herein, the terms “stem cell” or “stem cells” are used interchangeably to refer to undifferentiated cells (meaning cells having no specialized, i.e., mature, structure or function) having high proliferative potential with the ability to self-renew that can migrate to areas of injury and can generate daughter cells that can undergo terminal differentiation into more than one distinct cell phenotype.

The terms “variants”, “mutants” and “derivatives” of particular sequences of nucleic acids refer to nucleic acid sequences that are closely related to a particular sequence but which may possess, either naturally or by design, changes in sequence or structure. By “closely related”, it is meant that at least about 60%, but often, more than 85%, of the nucleotides of the sequence match over the defined length of the nucleic acid sequence. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce protein variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan.

As used herein, the terms “vector” and “expression vector” refer to a replicon, i.e., any agent that acts as a carrier or transporter, such as a phage, plasmid, cosmid, bacmid, phage or virus, to which another genetic sequence or element (either DNA or RNA) may be attached so as to bring about the replication of the attached sequence or element and so that sequence or element can be conveyed into a host cell.

It must be noted that as used herein and in the appended claims, the singular forms “a”, “and”, and “the” include plural referents unless the context clearly dictates otherwise. All technical and scientific terms used herein have the same meaning.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Materials and Methods

Plasmids

Cloning techniques generally may be found in J. Sambrook and D. W. Russell, Molecular Cloning: A Laboratory Manual, Third Edition, Cold Spring Harbor Press, Cold Spring Harbor, N.Y. (2001), which is incorporated herein by reference. Tetracycline-inducible MazF expression plasmid, pcDNA4/TO/mazF, was constructed by insertion of the MazF-coding region into the HindIII and EcoRI site of the pcDNA4/TO vector (Invitrogen, Carlsbad, Calif.). The MazE expression plasmid, pcDNA3/mazE, was created by ligation of the MazE-coding region and the pcDNA3 vector (Invitrogen, Carlsbad, Calif.) digested with EcoRI and KpnI. The pcDNA6/His/LacZ plasmid was purchased from Invitrogen (Carlsbad, Calif.).

Cell Lines

Immortalized baby mouse kidney (“iBMK”) cell line (W2), and the isogenic bax^{-/-} and bak^{-/-} (D3), bax^{-/-} (X2) and bak^{-/-} (K1) cell lines (Degenhardt, K., et al., Cancer Cell 2: 193-203 (2002); Degenhardt, K., et al., J. Biol. Chem. 277: 14127-14134 (2002)) were grown in DMEM (Gibco, Carlsbad, Calif.) supplemented with 5% fetal bovine serum (FBS) at 38.5° C. To establish human embryonic kidney cell lines (T-Rex-293) that stably express MazF alone or that co-express MazE with MazF, T-Rex-293 cells (Invitrogen, Carlsbad, Calif.) stably transfected with a tetracycline repressor expression plasmid (pcDNA6/TR) were co-transfected with pcDNA4/TO/mazF and pcDNA3 or pcDNA4/TO/mazF and pcDNA3/mazE by PolyFect Transfection Reagent (QIAGEN Inc, Valencia, Calif.) according to manufacture's instructions and were selected as follows: pcDNA6/TR; 5 μg/ml blasticidin (Invitrogen, Carlsbad, Calif.), pcDNA4TO/mazF; 40 μg/ml zeocin (Invitrogen, Carlsbad, Calif.) and pcDNA3/mazE; 0.5 μg/ml geneticin (Invitrogen, Carlsbad, Calif.) and ring cloning. One individual clone was selected for analysis. Two cell lines, T-Rex-293 (mazF/pcDNA3) and T-Rex-293 (mazF/mazE) were maintained in 10% calf serum-DMEM containing 40 μg/ml of zeocin, 5 μg/ml of blasticidin and 0.5 μg/ml of geneticin.

Trypan Blue Exclusion

The viability of T-Rex-293 cells was determined by trypan blue exclusion as previously described (Degenhardt, K., et al., J. Biol. Chem. 277: 14127-14134 (2002)). T-Rex-293 (mazF/pcDNA3) and T-Rex-293 (mazF/mazE) cells were cultured for 24 hr before tetracycline treatment and then incubated for 0, 24, 48 and 72 hr at 37° C. in the presence or absence of 10 μg/ml tetracycline. After treatment, cells were collected by centrifugation of the supernatant plus adherent cells, which were harvested by trypsinization. Cells were resuspended in 500 μl of fresh medium containing 0.1% trypan blue solution (Sigma, St. Louis, Mo.) and then counted on a hemocytometer to assess the number of dead blue cells and the total number of cells counted. As a control for apoptotic cell death, T-Rex-293 (mazF/mazE) cells were treated with 1 μM staurosporine (Sigma, St. Louis, Mo.) for same time periods concurrently with tetracycline treatment.

β-Galactosidase Assay

β-galactosidase assays to determine the viability of iBMK cells co-expressing LacZ and MazF were performed as previously described (Han, J., Sabbatini, P., and White, E. Mol. Cell. Biol. 16: 5857-5864 (1996)). W2, D3, X2 and K1 co-transfected with pcDNA6/His/LacZ and pcDNA4/TO/mazF or pcDNA3 for 48 hr were fixed in 1% glutaraldehyde and then stained with 0.2% X-gal solution at 37° C. for 24 hr. β-Galactosidase positive blue cells and the total number of cells (approximately 200 cells) were independently counted on a 6-cm diameter dish.

MTT Assay

3-(4,5-dimethylthiazol-2-yl)-2,5-diphenyltetrazolium bromide (MTT) assay to measure the viability of iBMK cells treated with paclitaxel, CHX, or both mouse TNF-α and CHX was performed as previously described (Ioffe, M. L., et al., Prostate 61: 243-247. (2004)).

Florescent Activated Cell Sorter (FACS) Analysis

T-Rex-293 (mazF/pcDNA3) cells and T-Rex-293 (mazF/mazE) cells treated with tetracycline for 0, 24, 48 and 72 hr were harvested by trypsinization, pelleted by centrifugation and resuspended in PBS. The cells were fixed with ice-cold 70% ethanol for 30 min and stained with propidium iodide (10 μg/ml) and RNase A (100 μg/ml) overnight. The cells were analyzed on a Becton Dickinson FACSCalibur system (San Jose, Calif.).

Western Blotting

Western blot analysis and immunofluorescence were performed as previously described (Perez, D., and White, E. J. Cell Biol. 141: 1255-1266 (1998); Perez, D., and White, E. Mol. Cell 6: 53-63 (2000)). The term “Western blot” refers to a method for identifying proteins in a complex mixture; proteins are separated electrophoretically in a gel medium; transferred from the gel to a protein binding sheet or membrane; and the sheet or membrane containing the separated proteins exposed to specific antibodies which bind to, locate, and enable visualization of protein(s) of interest. T-Rex-293 (mazF/pcDNA3) and T-Rex-293 (mazF/mazE) cells were harvested at 0, 24, 48 and 72 hr post-treatment with tetracycline or staurosporine by scraping and centrifugation. All cell pellets were lysed in 2× Laemmli buffer. Cell lysates were subjected to electrophoresis on a 15% SDS-PAGE gel and then transferred onto an Immobilon-P membrane (Millipore, Bedford, Mass.). The blot was incubated with following antibody: anti-active caspase-3 rabbit polyclonal antibody (Cell Signaling Technology, Beverly, Mass.), anti-BAX, anti-BAK rabbit polyclonal antibody (NT) (Upstate Biotechnology Inc., Lake Placid, N.Y.), anti-BID goat polyclonal antibody (R&D Systems, Inc, Minneapolis, Minn.), anti-Bim rabbit polyclonal antibody (Alexis Biochemical, San Diego, Calif.), anti-BCL-2 hamster monoclonal antibody (PharMingen, San Diego, Calif.), anti-BCL-X_{L} mouse monoclonal antibody (Trevigen, Gaithersburg, Md.), anti-MCL-1 rabbit polyclonal antibody (Stressgen Biotechnologies, Victoria, British Columbia, Canada), anti-PARP mouse monoclonal antibody (PharMingen, San Diego, Calif.), anti-PUMA rabbit polyclonal antibody (Nelson, D. A., et al., Genes Dev. 18: 2095-2107 (2004)), anti-E1A monoclonal antibody; anti-actin monoclonal antibody (Oncogene Research Products); and anti-Xpress monoclonal antibody (Invitrogen). An antibody directed to human NBK/BIK was generated by expression of a GST-tagged human NBK/BIK fusion protein encoding an N-terminal 78-amino acid region in bacteria and immunization of rabbits (Cocalico). Western Blots were developed with horseradish peroxidase-conjugated secondary antibodies using the ECL system (Amersham-Pharmacia Biotech, Piscataway, N.J.).

Immunofluorescence

W2, D3, X2 and K1 cells co-transfected by electroporation with a combination of plasmids pcDNA6/His/lacZ with pcDNA4/TO/mazF or pcDNA3 were grown on glass coverslips for 24 hr and fixed in 100% methanol for 10 min at −20° C. After blocking in 1% BSA-PBS for 1 hr at 37° C., cells on coverslips were incubated with anti-active caspase-3 rabbit polyclonal antibody and anti-Xpress mouse monoclonal antibody (Invitrogen, Carlsbad, Calif.) for 1 hr at 37° C. followed by incubation with rhodamine-conjugated anti-rabbit IgG and FITC-conjugated anti-mouse IgG antibody for 1 hr at 37° C. Staining was visualized by a Nikon FXA microscope equipped with epifluorescence optics (Nikon Inc., Garden City, N.Y.). The percentage of cells positive for active caspase-3 was determined by scoring 50-100 cells on each coverslip.

RNA Analysis

Northern blot analysis and real-time PCR (RT-PCR) was preformed as previously described (Zhang, Y., et al., Mol. Cell 12: 913-923 (2003); Cuconati, A., et al., Genes Dev. 17: 2922-2932 (2003)). Total RNA from tetracycline-treated (0, 24, 48 and 72 hr) or untreated (0, 24, 48 and 72 hr) T-Rex-293 (mazF/pcDNA3) and T-Rex-293 (mazF/mazE) cells for were isolated through the use of Trizol Reagent (Gibco, Carlsbad, Calif.). For Northern blot analysis, 10 μg of total RNA from each condition was subjected to formaldehyde-agarose gel electrophoresis followed by transfer onto GeneScreen Plus membrane (NEN Life Science Products, Boston, Mass.). The blot was hybridized with ³²P-labeled human GAPDH or β-actin cDNA. One hundred ng of total RNA was subjected to real time RT-PCR with the Taqman EZ-RT PCR kit (PE Applied Biosystems, Lincoln Centre Drive Foster City, Calif.) using recommended conditions provided by the vendor. Primers to analyze the level of human GAPDH mRNA were obtained from Applied Biosystems, and a probe for GAPDH was supplied with 5′ linkage to the reporter dye 2, 7-dimethoxy-4, 5-dichloro-6-carboxy-fluoroecein (JOE), and 3′ linkage to the quencher 6-carboxy-tetramethylrhodamine (TAMURA). β-actin primer sequences were 5′-GGGGAAATCGTGCGTGACATT-3′ and 5′-CGGATGTCCACGTCACACTT-3′. β-actin probe sequence was FAM-5′-ATCACCATTGGCAATGAGCGG TTCC-3′-TAMURA. Reactions were carried out in triplicate and the relative amount of human GAPDH and β-actin mRNA was calculated from the difference in fluorescence signal in the 24-, 48- and 72-hr samples compared with the corresponding 0 hr sample.

³⁵S-Methionine Metabolic Labeling

T-Rex-293 (mazF/pcDNA3) and T-Rex-293 (mazF/mazE) cells were incubated for 0, 24 and 48 hr in tetracycline-containing complete DMEM followed by incubation for 1 hr in fresh methionine-free DMEM containing 10 μCi/ml ³⁵S-methionine. The cells then were washed with PBS twice and lysed in 2× Laemmli buffer. The cell lysate was subjected to SDS-PAGE followed by autoradiography and coomassie blue stain. Signal intensity was measured by Phosphoimager STORM 860 (Molecular Dynamics, Sunnyvale, Calif.).

### Example 2

### MazF Induces Degradation of Cellular mRNA in Mammalian Cells

In E. coli, MazF functions as an mRNA interferase to cleave cellular mRNA, while the antitoxin MazE antagonizes the endoribonuclease activity of MazF (Zhang, Y., et al., Mol. Cell 12: 913-923 (2003)). Prompted by these findings, the question of whether MazF functions as an endoribonuclease in mammalian cells was explored. To this end, a tetracycline (Tet)-inducible MazF expression system in T-Rex-293 cells stably expressing the Tet repressor was developed through stable co-transfection of the Tet-inducible MazF expression plasmid with pcDNA3 or the constitutive MazE expression plasmid. RT-PCR with specific primers for a MazF- or MazE-coding region demonstrated Tet-dependent expression of MazF mRNA, and constitutive expression (meaning the gene is expressed continually with no control over its expression) of MazE in established T-Rex-293 cell lines (data not shown). Using this system, it was first examined whether cellular mRNAs were degraded in mammalian cells upon induction of MazF expression.

To assess cellular mRNA levels, total RNA was isolated from whole cells of Tet-treated or -untreated T-Rex-293 cells and subjected to Northern blot analysis using human GAPDH and β-actin cDNA probes. The term “Northern blot” as used herein refers to a technique in which RNA from a specimen is separated into its component parts on a gel by electrophoresis and transferred to a specifically modified paper support so that the mRNA is fixed in its electrophoretic positions. Labeled single-stranded DNA fragments complementary to the specific mRNA being sought then are hybridized to the bound mRNA and the label detected by suitable means. GAPDH and β-actin mRNAs were chosen as targets of MazF, since GAPDH and β-actin genes are known to be housekeeping genes, both mRNAs exist abundantly, and both are stable under diverse conditions. In addition, GAPDH and β-actin mRNAs have 20 and 22 ACA sequences in their respective protein-coding regions, which are targets for MazF cleavage (Zhang, Y., et al., Mol. Cell 12: 913-923 (2003)).

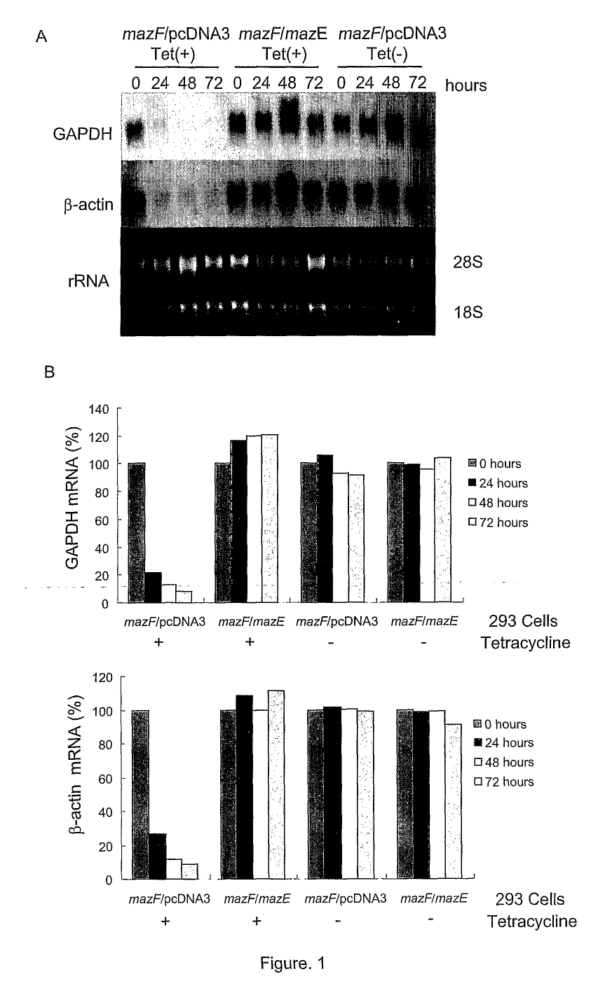

As shown in FIG. 1, MazF expression causes degradation of cellular mRNAs in mammalian cells. Panel (A) shows Northern blot analysis of human GAPDH and β-actin. Total RNA from Tet-treated or -untreated T-Rex-293 cells at the indicated time points was probed with ³²P-labeled human GAPDH and β-actin cDNA. 28S and 18S ribosomal RNAs were visualized by agarose-formaldehyde gel electrophoresis followed by ethidium bromide staining. Panel (B) shows quantification of mRNA levels. Human GAPDH and β-actin mRNA levels were quantified by real-time RT-PCR. Relative amounts of mRNA were calculated from the fluorescence signal in the 24-, 48- and 72-hours samples as compared with the corresponding 0-hour sample.

The data revealed that levels of both GAPDH and β-actin mRNAs were dramatically decreased in T-Rex-293 (mazF/pcDNA3) cells after 24 hours, and were almost completely lost by 48 hours post-induction of MazF (FIG. 1A). In contrast, levels of both mRNAs were maintained by co-expression of MazE with MazF throughout the time course of induction, similarly to the Tet-untreated control (FIG. 1A). The same results were obtained by real-time RT-PCR analysis using specific primers to amplify sequences of human GAPDH or β-actin cDNAs, each containing two ACA sequences (FIG. 1B). The data revealed a striking decrease (>90%) of both mRNAs upon MazF induction for 72 hours, whereas constant levels of both mRNAs were maintained in cells expressing both MazE and MazF, similarly to Tet-untreated control. Furthermore, it was notable that levels of 28S and 18S ribosomal RNA did not change even when MazF was expressed for 72 hours (FIG. 1A), indicating that ribosomal RNA interacting with ribosomal proteins in cells may be protected from degradation by MazF. Taken together, these data indicate that MazF specifically eliminates ACA sequence-containing cellular mRNA but not ribosomal RNA, and that MazE neutralizes MazF endoribonuclease function in mammalian cells as found for E. coli cells (Zhang, Y., et al., Mol. Cell 12: 913-923 (2003)).

### Example 3

### MazF Inhibits Protein Synthesis in Mammalian Cells

In E. coli cells, MazF induction causes protein synthesis to be inhibited through degradation of cellular mRNA, indicating that MazF is a general inhibitor for protein synthesis (Zhang, Y., et al., Mol. Cell 12: 913-923 (2003)). Thus, the effect of MazF on protein synthesis in T-Rex 293 cells was investigated.

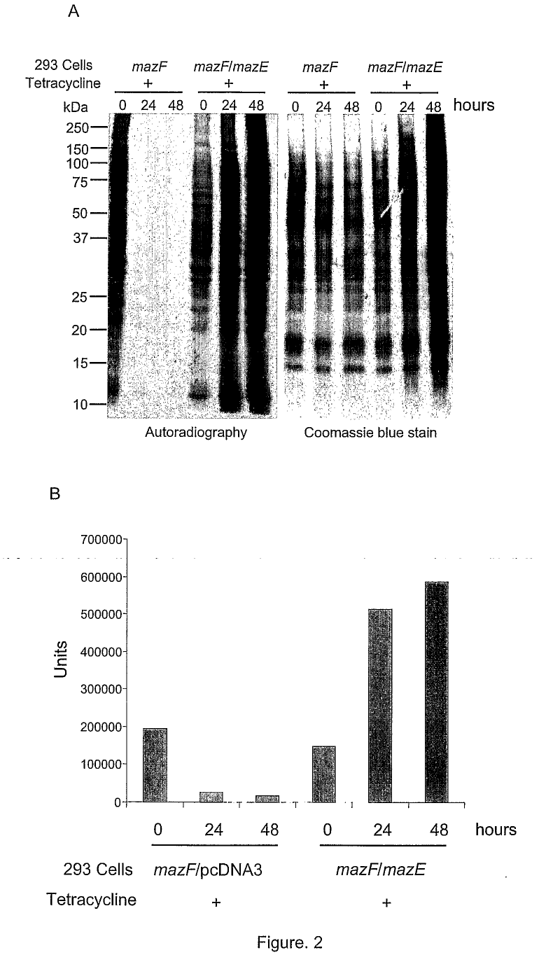

As shown in FIG. 2, MazF inhibits protein synthesis in mammalian cells. Panel (A) shows ³⁵S-methionine incorporation in T-Rex-293 cells. ³⁵S-methionine-labeled total protein from Tet-treated T-Rex-293 cells at the indicated time points was subjected to SDS-PAGE and autoradiography (left) and stained with Coomassie blue (right). Panel (B) shows quantification of ³⁵S-methionine-labeled proteins. Protein bands on the gel in panel (A) were scanned by Phosphoimager STORM 860 (Molecular Dynamics) and signal intensity was calculated.

SDS-PAGE analysis of whole cellular protein from Tet-treated T-Rex-293 (mazF/pcDNA3) and T-Rex-293 (mazF/mazE) cells evaluated for ³⁵S-methionine incorporation at 0, 24 and 48 hours post induction of MazF demonstrated that protein synthesis was strikingly inhibited as rapidly as 24 hours (FIG. 2A). In contrast, co-expression of MazE clearly prevented the inhibitory effect of MazF on protein synthesis (FIGS. 2A and 2B). Furthermore, the loss of ³⁵S-methionine incorporation was not due to an overall loss of cellular protein in MazF expressing cells as total protein levels remain constant over the 48 hour induction period (FIG. 2A). The data indicate that MazF functions as an inhibitor of protein synthesis and MazE represses inhibition of protein synthesis by MazF in mammalian cells as found in E. coli cells (Zhang, Y., et al., Mol. Cell 12: 913-923 (2003)).

### Example 4

### MazF Induces Apoptotic Cell Death in Mammalian Cells

In E. coli, sequence-specific mRNA interference by MazF leads to rapid cell growth arrest and eventual cell death (Zhang, Y., et al., Mol. Cell 12: 913-923 (2003)). Thus, the impact of MazF expression, mRNA elimination, and inhibition of protein synthesis on mammalian cell proliferation and viability was examined.

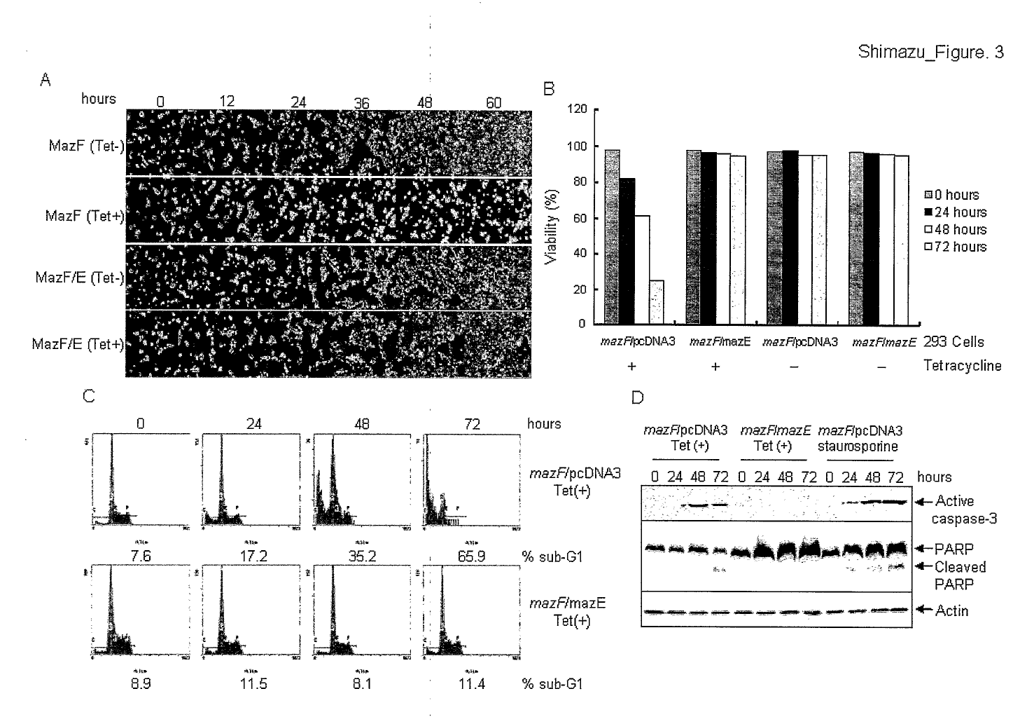

FIG. 3 shows that MazF induces apoptotic cell death in mammalian cells. Panel (A) shows phase contrast photographs of Tet-treated or -untreated T-Rex-293 cells (magnification 100×). Panel (B) shows a viability analysis of T-Rex-293 cells in (A). Tet-treated or -untreated T-Rex-293 cells at the indicated time points were subjected to trypan blue exclusion. Viability was represented as a percent of total cells at time 0. Panel (C) is a representative illustration of propidium iodide labeling measured by FACS in Tet-treated T-Rex-293 cells. Panel (D) is a Western blot analysis of lysates from T-Rex-293 cells. Whole cell lysates from Tet-treated or staurosporine-treated T-Rex-293 cells at the indicated time points were immunoblotted with an anti-active caspase-3 antibody (top), anti-PARP antibody (middle) and anti-actin antibody (bottom).

Induction of MazF in T-Rex-293 halted cell accumulation and induced a progressive cytopathic effect (CPE) (meaning degenerative changes in cells) during the time course of MazF induction (FIG. 3A). Notably, the number of attached cells was dramatically decreased at 72 hours post-induction of MazF (FIG. 3A). In contrast, in cells where MazE was co-expressed with MazF, cell number and morphology were maintained similarly to cells without MazF induction (mazF/pcDNA3 Tet (−) and mazF/mazE Tet (−)) (FIG. 3A). Thus, MazE is capable of neutralizing the toxic effect of MazF on mammalian cells.

The viability of T-Rex-293 cells that express MazF also was quantified by trypan blue exclusion. When MazF expression was induced, the cell viability of T-Rex-293 (mazF/pcDNA3) cells dropped strikingly (FIG. 3B). In contrast, co-expression of MazE with MazF conferred resistance to MazF-mediated killing (FIG. 3B) and cells without MazF or MazE alone also remained viable (FIG. 3B). Viability was also analyzed by fluorescence-activated cytometry for DNA content, and these results showed the same trends as trypan blue exclusion with apoptotic cell death by MazF induction indicated by accumulation of a sub G1 peak. The sub-G1 peak in T-Rex-293 (mazF/pcDNA3) increased in time-dependent manner of Tet treatment, up to 65.9% at 72 hours of induction, whereas, the sub-G1 peak in T-Rex-293 (mazF/mazE) cells remained low (11.4% at 72 hours of induction) (FIG. 3C). These data demonstrate that MazF toxin induces cell death and that MazE antitoxin prevents MazF-dependent cell death in mammalian cells.

MazF-induced time-dependent induction of cell death was consistent with the occurrence of MazF-induced apoptosis in human 293 cells. To confirm that cell death induced by MazF was apoptosis, we examined whether caspase-3, one of the executioner caspases in the apoptosis pathway, was activated in T-Rex-293 cells expressing MazF. Western blot analysis using an antibody that recognizes cleaved and activated caspase-3 revealed the presence of the processed active form of caspase-3 in extracts from T-Rex-293 (mazF/pcDNA3) cells treated with Tet (FIG. 3D). Active caspase-3 was also detected in extracts from T-Rex-293 (mazF/mazE) cells treated with staurosporine, a known inducer of apoptosis (FIG. 3D). In contrast, activation of caspase-3 was inhibited by co-expression of MazE with MazF (Tet-treated T-Rex-293 (mazF/mazE) cells, FIG. 3D). In addition, we also examined cleavage of PARP, a substrate of activated caspase-3. Cleaved PARP was detected in extracts from T-Rex-293 (mazF/pcDNA3) cells treated with Tet for 48 hours and the levels further increased at 72 hours post-induction of MazF, similarly to staurosporine-treated T-Rex-293 (mazF/mazE) cells. As expected, in extracts of cells expressing both MazE and MazF, where the processed active caspase-3 was not detected, cleaved PARP was also not present (FIG. 3D). Taken together, the data clearly demonstrate that MazF toxin, a sequence-specific endoribonuclease from E. coli, induces apoptotic cell death in mammalian cells, which can be prevented by MazE antitoxin co-expression.

### Example 5

### Levels of BCL-2 Family Proteins do not Change During MazF-Induced Apoptosis

To gain insight into the mechanism of apoptosis induction by MazF upstream of caspase-3 activation, anti-apoptotic (BCL-2, BCL-X_{L} and MCL-1) or proapoptotic (BAK, BAK, BID, BIM, NBK/BIK and PUMA) protein levels were examined for modulation by MazF-mediated mRNA cleavage. Cell lysates from Tet-treated T-Rex-293 (mazF/pcDNA3) and T-Rex-293 (mazF/mazE) cells for 0, 24, 48 and 72 hours were subjected to Western blot analysis.

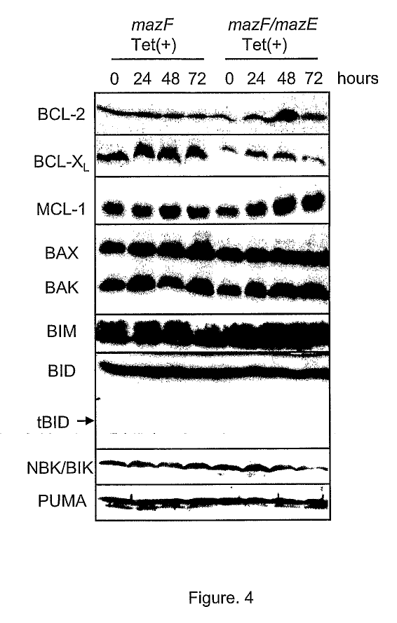

FIG. 4 shows that levels of BCL-2 family proteins remain constant during MazF induction. Whole cell lysates from Tet-treated T-Rex-293 cells were immunoblotted with antibodies that specifically recognize anti-apoptotic and proapoptotic proteins indicated in the figure. The levels of BCL-2 family proteins remained unchanged and truncated BID (tBID) was also undetectable during MazF-induced apoptosis. These data suggest that loss of anti-apoptotic BCL-2 family members, BCL-2, BCL-X_{L}, and MCL-1 or upregulation of proapoptotic BAX, BAK, BIM, BID, NBK/BIK, and PUMA proteins were not responsible for MazF-mediated apoptosis. Finally, the absence of tBID in MazF expressing cells suggests that the apoptotic pathway mediated by death receptor via tBID also is not involved.

### Example 6

### MazF-Induced Apoptosis Requires BAK but not BAX

Proapoptotic members of the BCL-2 family, BAX and BAK play crucial but predominantly functionally redundant roles in the mitochondria-dependent apoptosis pathway induced by numerous apoptotic stimuli downstream of BH3-only proteins (Danial, N. N., and Korsmeyer, S. J., Cell 116: 205-219 (2004); Gelinas, C., and White, E. (2005). Genes Dev. 19: 1263-1268 (2005); Tsujimoto, Y., J. Cell Physiol. 195: 158-67 (2003); Willis, S. N., and Adams, J. M. Curr. Opin. in Cell Biol. 17: 1-9 (2005)). To test the potential involvement of BAX and/or BAK in MazF-mediated apoptosis, advantage was taken of W2 (bax^{+/-}bak^{+/+}), D3 (bax^{-/-}bak^{-/-}), X2 (bax^{-/-}bak^{+/-}) and K1 (bax^{+/-}bak^{-/-}) iBMK cells (Degenhardt, K., and White, E. Clin. Cancer Res. in press. (2006); Degenhardt, K., et al. Cancer Cell, 51-64 (2006); Degenhardt, K., et al., Cancer Cell 2: 193-203 (2002); Degenhardt, K., et al., J. Biol. Chem. 277: 14127-14134 (2002)).

Tumor necrosis factor-α (TNF-α) induces apoptosis in W2, X2, and K1 iBMK cells, whereas, D3 iBMK cells are resistant to TNF-α-induced apoptosis and to apoptosis mediated by many other stimuli (Degenhardt, K., et al., J. Biol. Chem. 277: 14127-14134 (2002b)). First, the MazF expression plasmid, pcDNA4/TO/mazF was transiently co-transfected with a lacZ expression plasmid, pcDNA6/His/lacZ into W2, D3, X2 and K1 cells and then a β-galactosidase assay was performed to monitor the impact of MazF transient expression.

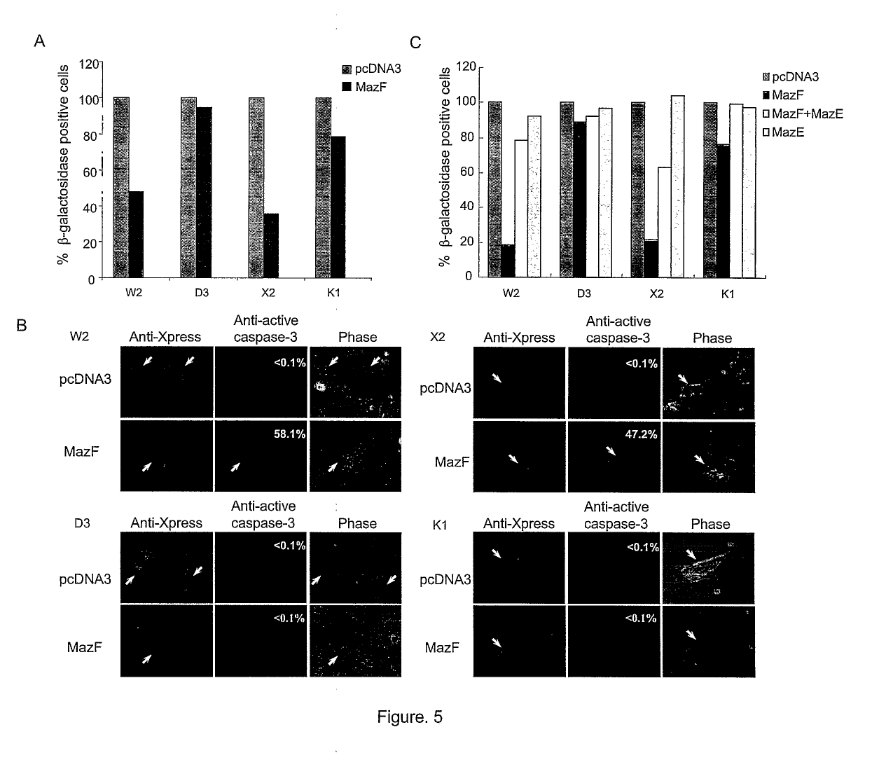

FIG. 5 shows that BAK function is required for MazF-induced apoptosis. Panel (A) shows viability of iBMK cells transiently expressing MazF. W2, D3, X2 and K1 cells transiently co-expressing LacZ and MazF were subjected to a β-galactosidase assay at 48 hours post-transfection. β-Galactosidase positive blue cells were calculated as its percentage of total cells. Panel (B) shows immunofluorescence of activated caspase-3 in iBMK cells. W2, D3, X2 or K1 cells transiently co-expressing LacZ and MazF were co-stained with anti-Xpress and anti-active caspase-3 antibody. FITC (green) and rhodamine (red) stain represent cells expressing LacZ and activated caspase-3, respectively. Numbers represents the percentage of activated-caspase-3 positive cells. White arrows indicate the corresponding activated-caspase-3 positive cells from the matching FITC-stained cells. Panel (C) shows viability of iBMK cells transiently co-expressing MazF and MazE. W2, D3, X2 and K1 cells transiently co-expressing LacZ and MazF and/or MazE were subjected to a β-galactosidase assay. β-Galactosidase positive blue cells were calculated as described above.

As shown in FIG. 5, MazF expression in W2 cells resulted in a significant decrease of β-galactosidase positive cells (FIG. 5A). In contrast, little effect on β-galactosidase expression was observed in D3 cells expressing MazF (FIG. 5A). Interestingly, the number of β-galactosidase positive cells in X2 cells was significantly decreased, similar to W2 cells, whereas K1 cells were preferentially resistant to MazF, suggesting that BAK deficiency was sufficient to tolerate MazF expression. Moreover, the preservation of β-galactosidase expression by MazF in K1 cells indicates that the loss of expression is due to apoptosis but not elimination of β-galactosidase mRNA.

To test whether the lack of β-galactosidase expression in W2 and X2 cells was due to apoptosis, MazF expressing cells were examined for active caspase-3. Immunofluoresence using anti-active caspase-3 antibody showed the presence of active-caspase-3 positive cells (red) in 58.1% of W2 cells and 47.2% of X2 cells transiently expressing MazF compared with that in <0.1% of pcDNA3-transfected W2 and X2 cells (FIG. 5B). However, D3 and K1 cells transiently expressing MazF had few cells with activated caspase-3 (<0.1%) (FIG. 5B), indicating that it was primarily the loss of BAK that prevented caspase-3 activation by MazF. Taken together, the data indicate that MazF induces a BAK- but not BAX-dependent mechanism(s) to activate caspase-3 and apoptosis.

To investigate whether MazE can suppress BAK-mediated apoptosis induced by MazF, MazE was transiently co-expressed in W2, D3, X2 or K1 cells with MazF. The result from the β-galactosidase assay showed that MazE expression in W2 and X2 cells with MazF significantly repressed MazF-induced cell death, similarly to W2 and X2 cells transfected with pcDNA3 or MazE expression plasmid alone (FIG. 5C).

### Example 7

### NBK/BIK is Required for Cell Death Induced by Inhibition of Protein Synthesis

To identify the pathway by which inhibition of protein synthesis triggers BAK-mediated apoptosis, the functional requirement for upstream BH3-only proapoptotic proteins was examined. iBMK cell lines deficient for individual BH3-only proapoptotic proteins (PUMA, BIM, NOXA and NBK/BIK) (Tan, T. T., et al., Cancer Cell 7: 227-238. (2005)) were tested for resistance to MazF-mediated apoptosis. MazF was transiently co-expressed with LacZ in puma^{-/-}, bim^{-/-}, noxa^{-/-} or nbk/bik^{-/-} iBMK cells and monitored for β-galactosidase activity.

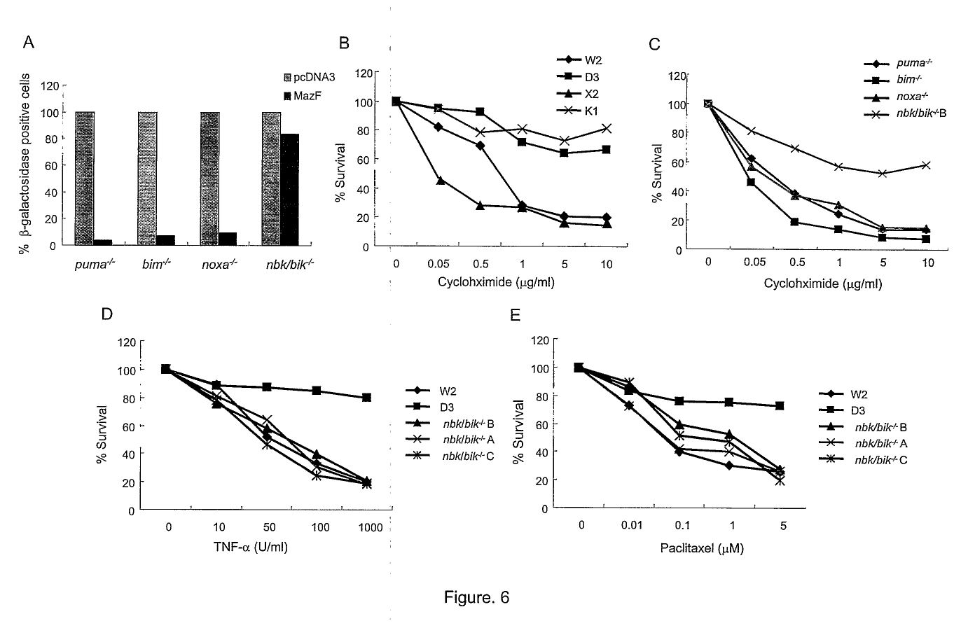

FIG. 6 shows that NBK/BIK mediates MazF or CHX-induced cell death. Panel (A) shows viability of iBMK cells transiently expressing MazF. nbk/bik^{-/-}, bim^{-/-}, noxa^{-/-} or puma^{-/-} iBMK cells co-expressing LacZ and MazF were subjected to a β-galactosidase assay. β-Galactosidase positive blue cells were calculated as a percentage of total cells. Panels (B) and (C) show viability of CHX-treated iBMK cells. W2, D3, X2 and K1 cells (panel B), and nbk/bik^{-/-}B, bim^{-/-}, noxa^{-/-} or puma^{-/-} cells (panel C) treated with CHX were subjected to an MTT assay. Panels (D) and (E) show viability of TNF-α/CHX- and paclitaxel-treated iBMK cells. W2, D3 and three independent nbk/bik^{-/-} cell lines (A, B and C) treated with TNF-α/CHX (0.05 μg/ml) (panel D) and paclitaxel (panel E) were subjected to an MTT assay.

FIG. 6 shows that the number of β-galactosidase positive cells in puma^{-/-}, bim^{-/-}, noxa^{-/-} cells transiently expressing MazF significantly decreased (FIG. 6A), similarly to W2 and X2 cells (FIG. 5A). Interestingly, nbk/bik^{-/-} cells were preferentially resistant to MazF-induced cell death (FIG. 6A), similarly to D3 and K1 cells (FIG. 5A). This suggests that MazF-mediated apoptosis requires NBK/BIK that signals through BAK.

To test if apoptosis mediated by general translation inhibition was dependent on specific BH3-only proteins, the apoptotic response of puma^{-/-}, bim^{-/-}, noxa^{-/-}, nbk/bik^{-/-} iBMK cells to translation inhibition was tested with CHX. In addition to W2, D3, X2 or K1 cells, puma^{-/-}, bim^{-/-}, noxa^{-/-} or nbk/bik^{-/-} cells were treated with increasing concentration of CHX (0.05, 0.5, 1.0, 5.0 and 10.0 μg/ml) for 24 hours to induce translation inhibition and viability was assessed. The survival of W2, X2, puma^{-/-}, bim^{-/-} and noxa^{-/-} cells treated with CHX decreased in a dose-dependent manner (FIGS. 6B and C). In contrast, when D3, K1 (FIG. 6B) and nbk/bik^{-/-} (FIG. 6C) cells were treated with CHX, cell viability remained high. In addition, W2 and three independent nbk/bik^{-/-} iBMK cell lines were clearly sensitive to TNF-α-(FIG. 6D) and paclitaxel-induced cell death (FIG. 6E). In contrast, D3 cells were resistant to cell death induced by TNF-α and paclitaxel (FIGS. 6D and E). These results clearly indicate that NBK/BIK is the BH3-only proapoptotic protein that is required for cell death incurred by inhibition of protein synthesis in response to MazF-induced mRNA degradation and CHX-induced translation inhibition upstream of BAK. Furthermore, NBK/BIK was not required for TNF-α-mediated apoptosis (FIG. 6D) which signals through tBID (Li, H., et al., Cell 94: 491-501 (1998); Luo, X., et al., Cell 94: 481-490 (1998)), nor was NBK/BIK required for apoptosis induced by taxanes (FIG. 6E) which is dependent on BIM (Bouillet, P., et al., Science 286: 1735-1738 (1999); Tan, T. T., et al., Cancer Cell 7: 227-238. (2005)). These findings support the role for specific BH3-only proteins controlling the response to discrete apoptotic stimuli.

### Example 8

### NBK/BIK is a Mediator of Apoptosis Induced by Adenovirus Infection

Productive adenovirus infection abrogates host cell protein synthesis and triggers induction of apoptosis. To investigate whether NBK/BIK is required for adenovirus-induced apoptosis concomitant with shutoff of host cell protein synthesis, W2, D3 and nbk/bik^{-/-} iBMK cells were infected with wild-type adenovirus type 5 (Ad5d/309) and an E1B 19K gene deletion (anti-apoptotic vBCL-2) mutant (Ad5d/337), and monitored for CPE.

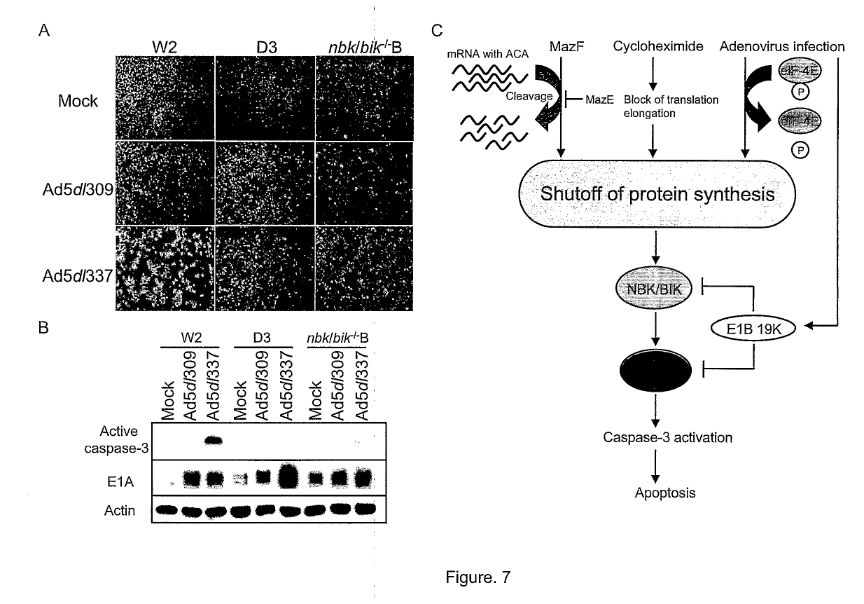

FIG. 7 shows that NBK/BIK mediates adenovirus-induced apoptosis. Panel (A) shows phase contrast photographs of adenovirus-infected iBMK cells (magnification 100×). Panel (B) shows Western blot analysis with lysates from viral infected iBMK cells. Whole cell lysates from mock-, Ad5d/309- or Ad5d/337-infected W2, D3 and nbk/bik^{-/-} B cells were immunoblotted with an anti-active caspase-3 antibody (top), anti-E1A antibody (middle) and anti-actin antibody (bottom). Panel (C) shows the apoptosis pathway induced by shutoff of protein synthesis.

Cell morphology of Ad5d/309-infected W2, D3 and nbk/bik^{-/-} cells was maintained, similar to that of mock-infected W2, D3 and nbk/bik^{-/-} cells, indicating that E1B 19K prevents adenovirus-induced apoptosis (FIG. 7A). Infection with Ad5d/337 into W2 cells resulted in almost complete destruction of the monolayer at 48 hours post-infection indicative of apoptosis (FIG. 7A) as expected (Cuconati, A., et al., J. Virol. 76: 4547-4558 (2002)). In contrast, Ad5d/337-infected D3 cells were resistant to adenovirus-induced apoptosis (FIG. 7A) as expected (Cuconati, A., et al., J. Virol. 76: 4547-4558 (2002)). Interestingly, apoptotic CPE was not observed in Ad5d/337-infected nbk/bik^{-/-} cells (FIG. 7A).

Apoptosis of infected iBMK cells was assessed by monitoring activation of caspase-3. As expected, in W2 and D3 cells infected with Ad5d/309, the processed active form of caspase-3 was undetectable (FIG. 7B). The activated caspase-3 was also not seen in nbk/bik^{-/-} cells infected with Ad5d/309 (FIG. 7B). Abundant levels of activated caspase-3 were present in extracts from Ad5d/337-infected W2 cells, whereas activated caspase-3 did not appear in extracts from Ad5d/337-infected D3 cells (FIG. 7B), as expected (Cuconati, A., et al., J. Virol. 76: 4547-4558 (2002)). Interestingly, the amount of activated caspase-3 detected in nbk/bik^{-/-} cells infected with Ad5d/307 was significantly less than that detected in Ad5d/307-infected W2 cells (FIG. 7B), which was consistent with the resistance of nbk/bik^{-/-} cells to Ad5d/337-induced cell death (FIG. 7A). To ensure that iBMK cells were infected and expressing viral proteins, cell lysates were analyzed for E1A protein levels. The term “E1A” as used herein refers to an adenovirus protein that affects cellular functions by binding to and sequestering cellular proteins, thereby preventing them from taking part in cellular processes. Since E1A was used to immortalize the iBMK cells, low levels of E1A protein expression were detected in uninfected cells (FIG. 7B). E1A levels increased significantly in Ad5d/309-infected W2, D3 and nbk/bik^{-/-} cells (FIG. 7B), indicating that virally infected cells expressed E1A from the viral genome. Higher levels of E1A were observed in Add/337-infected D3 and nbk/bik^{-/-} cells (FIG. 7B), indicating that production of E1A in Add/337-infected D3 and nbk/bik^{-/-} cells was due to absence of apoptosis by deficiency of BAX, BAK and NBK/BIK. Thus, these data indicate that NBK/BIK is a mediator that regulates apoptosis induced by adenovirus infection upstream of BAX and BAK.

While the present invention has been described with reference to the specific embodiments thereof, it should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the present invention. All such modifications are intended to be within the scope of the claims appended hereto.

1. A method of selectively regulating apoptosis in a mammalian cell, the method comprising the steps:
a. preparing a first expression vector comprising an isolated nucleic acid sequence encoding an mRNA interferase polypeptide, a derivative of the mRNA interferase polypeptide, or a fragment of the mRNA interferase polypeptide;
b. preparing a second expression vector comprising an isolated nucleic acid sequence encoding an mRNA interferase antagonist polypeptide, a derivative of the mRNA interferase antagonist polypeptide, or a fragment of the mRNA interferase antagonist polypeptide;
c. introducing the first expression vector into the mammalian cell,
d. optionally introducing the second expression vector into the mammalian cell;
e. selectively inducing expression of the first expression vector encoding the mRNA interferase polypeptide, the derivative of the mRNA interferase polypeptide, or the fragment of the mRNA interferase polypeptide, thereby inducing apoptosis in the cell, and
f. optionally selectively inducing expression of the second expression vector encoding the mRNA interferase antagonist polypeptide, the derivative of the mRNA interferase antagonist polypeptide, or the fragment of the mRNA interferase antagonist polypeptide, thereby inhibiting apoptosis in the cell.

2. The method according to claim 1, wherein the first expression vector and the second expression vector each further comprise at least one regulatory sequence.

3. The method according to claim 2, wherein the at least one regulatory sequence is at least one inducible promoter.

4. The method according to claim 3, wherein the at least one inducible promoter in the first expression vector is operably linked to the nucleic acid sequence encoding the mRNA interferase polypeptide, the derivative of the mRNA interferase polypeptide, or the fragment of the mRNA interferase polypeptide.

5. The method according to claim 3, wherein the at least one inducible promoter in the second expression vector is operably linked to the nucleic acid sequence encoding the mRNA interferase antagonist polypeptide, the derivative of the mRNA interferase antagonist polypeptide, or the fragment of the mRNA interferase antagonist polypeptide.

6. The method according to claim 1 wherein the mRNA interferase polypeptide, derivative of the mRNA interferase polypeptide, or fragment of the mRNA interferase polypeptide, when expressed in the cell, recognizes an at least one first mRNA interferase recognition sequence in cellular messenger RNA.

7-11. (canceled)

12. The method according to claim 1, wherein the target mammalian cell is Bak-deficient.

13. The method according to claim 1, wherein the target mammalian cells is Bak- and Bax-deficient.

14-22. (canceled)

23. A method of maintaining an isolated nucleic acid sequence encoding an mRNA interferase polypeptide, a derivative of the mRNA interferase polypeptide, or a fragment of the mRNA interferase polypeptide in a mammalian cell, the method comprising the steps:
a. preparing an expression vector comprising an isolated nucleic acid sequence encoding an mRNA interferase polypeptide, a derivative of the mRNA interferase polypeptide, or a fragment of the mRNA interferase polypeptide; and
b. introducing the expression vector into the mammalian cell, wherein at least one apoptotic pathway of the mammalian cell is blocked.

24. The method according to claim 23, the method further comprising the step of
c. inducing the expression of the mRNA interferase polypeptide, the derivative of the rRNA interferase polypeptide, or a fragment of the mRNA interferase polypeptide after step b.

25. The method according to claim 23, wherein the expression vector further comprises at least one regulatory sequence.

26. The method according to claim 25, wherein the at least one regulatory sequence is at least one inducible promoter which is operably linked to the nucleic acid sequence encoding the mRNA interferase polypeptide, the derivative of the mRNA interferase polypeptide, or the fragment of the mRNA interferase polypeptide.

27. The method according to claim 23 wherein the mRNA interferase polypeptide, derivative of the mRNA interferase polypeptide, or fragment of the mRNA interferase polypeptide, when expressed in the cell, recognizes an at least one mRNA interferase recognition sequence in cellular messenger RNA.

28-30. (canceled)

31. The method according to claim 23, wherein the mammalian cells is BAK deficient, NBK/BIK deficient, or BAK deficient and NBK/BIK deficient.

32. A mammalian cell harboring an isolated nucleic acid sequence encoding an mRNA interferase polypeptide, a derivative of the mRNA interferase polypeptide, or a fragment of the mRNA interferase polypeptide, wherein at least one apoptotic pathway of the mammalian cell is blocked.

33. The mammalian cell according to claim 32, wherein the mammalian cell is transduced by an expression vector comprising an isolated nucleic acid sequence encoding an mRNA interferase polypeptide, a derivative of the mRNA interferase polypeptide, or a fragment of the mRNA interferase polypeptide.

34. The mammalian cell according to claim 33, wherein the expression vector further comprises at least one regulatory sequence.

35. The mammalian cell according to claim 34, wherein the at least one regulatory sequence is at least one inducible promoter which is operably linked to the nucleic acid sequence encoding the mRNA interferase polypeptide, the derivative of the mRNA interferase polypeptide, or the fragment of the mRNA interferase polypeptide.

36. The mammalian cell according to claim 32 wherein the mRNA interferase polypeptide, derivative of the mRNA interferase polypeptide, or fragment of the mRNA interferase polypeptide, when expressed in the cell, recognizes an at least one mRNA interferase recognition sequence in cellular messenger RNA.

37-39. (canceled)

40. The mammalian cell according to claim 32, wherein the mammalian cell is BAK deficient, NBK/BIK deficient, or BAK deficient and NBK/BIK deficient.


SEQUENCE LISTING (3 sequences)

SEQ ID NO: 1 - DNA, 20 bp, Artificial Sequence
  beta-actin primer sequence
gggaaatcgtgcgtgacatt

SEQ ID NO: 2 - DNA, 20 bp, Artificial Sequence
  beta-actin primer sequence
cggatgtccacgtcacactt

SEQ ID NO: 3 - DNA, 25 bp, Artificial Sequence
  beta-actin probe sequence
atcaccattggcaatgagcggttcc